(19) United States
(12) Patent Application Publication
(10) Pub. No.: US 2018/0117167 A1
(43) Pub. Date: May 3, 2018

(54) PROCESS FOR PREPARING POLYURETHANE FOAM DRESSING COMPRISING ANTI-INFLAMMATORY AGENT

(71) Applicant: DAEWOONG PHARMACEUTICAL CO., LTD., Seongnam-si, Gyeonggi-do (KR)

(72) Inventors: Hyeon-A YIM, Bucheon-si, Gyeonggi-do (KR); Hee-Chul CHANG, Seoul (KR)

(73) Assignee: DAEWOONG PHARMACEUTICAL CO., LTD., Seongnam-si, Gyeonggi-do (KR)

(21) Appl. No.: 15/568,266

(22) PCT Filed: Apr. 20, 2016
(86) PCT No.: PCT/KR2016/004084

§ 371 Date: Oct. 20, 2017

(30) Foreign Application Priority Data
Apr. 23, 2015 (KR) 10-2015-0056977

(51) Int. Cl. A61K 47/59; A61K 9/70; A61K 31/192; A61K 31/165; A61L 26/00

(52) U.S. Cl. CPC A61K 47/59; A61K 9/7015; A61K 9/7069; A61K 31/192; A61L 2300/41; A61L 26/0019; A61L 26/0066; A61L 26/0085; A61K 31/165

(57) ABSTRACT

Provided is a process for preparing a polyurethane foam dressing in which an anti-inflammatory agent is homogeneously dispersed, the process including obtaining a drug (i.e., anti-inflammatory agent)-containing polyurethane prepolymer using a certain dispersing agent; and reacting the drug-containing polyurethane prepolymer with a water-containing blowing solution to form a polyurethane foam and then drying the polyurethane foam.

### TECHNICAL FIELD

The present invention relates to a process for preparing a polyurethane foam dressing. More specifically, the present invention relates to a process for preparing a polyurethane foam dressing in which an anti-inflammatory agent is homogeneously dispersed.

### BACKGROUND ART

A wound is a skin injury in which skin is torn, cut or become open by external stimuli. It entails large amounts of exudate and pain due to skin incision and injuries. A wound dressing, one of the medical products for wound healing, is used for protecting wounds, preventing contamination, absorbing exudates, preventing bleeding or loss of body fluid, and so on. There have been developed various wound dressings for absorbing exudates and preventing bleeding, such as foam dressings, alginate dressings, hydrocolloid dressings, and hydrogel dressings. And also, in order to prevent infections due to secondary contamination, silver-containing dressings are being developed. However, because such wound dressings perform only the primary purposes, there is a limitation that they cannot perform the functions for improving the patients' conditions, such as pain relief associated with wounds.

A foam dressing reduces pain by maintaining a moist environment in the wound. In other words, a foam dressing absorbs the exudate generated by the wound, thereby maintaining the moist environment. Especially, because the wounds having deep and wide injuries lead to secretion of large amounts of exudate, there have been developed polyurethane foam dressings comprising an anti-inflammatory agent such as ibuprofen, in order to both absorb exudate and ameliorate pain.

Meanwhile, a drug-containing polyurethane foam dressing requires distributing the drug homogeneously therein, in order to accomplish proper and uniform anti-inflammatory and analgesic effects in the entire wound sites. Furthermore, because a drug-containing polyurethane foam dressing is cut into an appropriate size according to the wound size and then applied to the wound, a uniform drug distribution therein is essential for accomplishing desired anti-inflammatory and analgesic effects uniformly in the wound sites. In addition, it is required that the blowability and the exudate-absorbing capacity of a drug-containing polyurethane foam dressing are maintained in equivalent levels to those of a non-drug containing polyurethane foam dressing.

Conventional processes for preparing a polyurethane foam dressing comprise forming a polyurethane foam by reacting a polyurethane prepolymer containing a polyol and an isocyanate with a blowing solution containing a blowing agent (e.g., water) and a polymerization initiator. In order to obtain a polyurethane foam in which a drug such as an anti-inflammatory agent is uniformly dispersed, it is required to perform a step for adding and dispersing the drug homogeneously in the process. However, if a drug is added in the step for forming a polyurethane prepolymer, the resulting prepolymer shows too high viscosity and the drug cannot be sufficiently dissolved therein, which makes it difficult to obtain a polyurethane foam in which the drug is uniformly dispersed. And also, if a drug is added to a blowing solution, the drug is precipitated out from the blowing solution, which also makes it difficult to obtain a polyurethane foam in which the drug is uniformly dispersed. And also, addition of a solubilizing agent (e.g., surfactants) for solubilizing a drug to a blowing solution brings about problems such as drug aggregation, reduced pore uniformity, and so on.

Therefore, there is a need in the art to develop a process for preparing a polyurethane foam dressing in which a drug such as an anti-inflammatory agent is homogeneously dispersed, wherein blowability and exudate-absorbing capacity of the polyurethane foam dressing can be effectively maintained.

### DISCLOSURE

### Technical Problem

The present inventors carried out various researches in order to develop a process for preparing a polyurethane foam dressing in which an anti-inflammatory agent is homogeneously dispersed. Especially, the present inventors have reviewed various dispersing agents which can disperse an anti-inflammatory agent in a polyurethane prepolymer and be easily removed in the process, while maintaining not only the blowability in the foam-forming step but also the exudate-absorbing capacity of the resulting polyurethane foam dressing. Surprisingly, the present inventors have found that, if a solution obtained by dissolving an anti-inflammatory agent in a certain dispersing agent such as ethanol is mixed with a polyurethane prepolymer to obtain a drug-containing polyurethane prepolymer, followed by reacting with a blowing solution to form a polyurethane foam and then drying the polyurethane foam, the blowability and the exudate-absorbing capacity of the resulting polyurethane foam dressing can be maintained; and the anti-inflammatory agent is homogeneously dispersed therein.

Therefore, the present invention provides a process for preparing a polyurethane foam dressing in which an anti-inflammatory agent is homogeneously dispersed, the process comprising mixing a solution obtained by dissolving an anti-inflammatory agent in the certain dispersing agent with a polyurethane prepolymer to obtain a drug-containing polyurethane prepolymer.

### Technical Solution

In accordance with an aspect of the present invention, there is provided a process for preparing a polyurethane foam dressing in which an anti-inflammatory agent is homogeneously dispersed, the process comprising: (a) mixing an alkaline anti-inflammatory agent having a pKa value more than pKa 7; one or more dispersing agents selected from the group consisting of methanol, ethanol, n-propanol, isopropanol, ethyl acetate and n-hexane; and a polyurethane prepolymer comprising a polyol and an isocyanate to obtain a drug-containing polyurethane prepolymer, and (b) reacting the drug-containing polyurethane prepolymer obtained in the step (a) with a blowing solution comprising water and a polymerization initiator to form a polyurethane foam and then drying the polyurethane foam.

In the process of the present invention, the step (a) may be performed by mixing a drug solution obtained by dissolving the anti-inflammatory agent in the dispersing agent with the polyurethane prepolymer. The anti-inflammatory agent may be acetaminophen, ibuprofen, or dexibuprofen, preferably dexibuprofen.

The dispersing agent may be used in an amount ranging from 0.2 to 20% by weight, based on the total weight of the polyurethane prepolymer. Preferably, the dispersing agent may be ethanol. In an embodiment, the step (a) may be performed by mixing a drug solution obtained by dissolving dexibuprofen in ethanol with the polyurethane prepolymer. For example, the drug solution may be obtained by dissolving dexibuprofen in ethanol in a concentration ranging from 0.005 to 50 g/ml.

In the process of the present invention, the polyol may be a copolymer of ethylene oxide and propylene oxide having 500 to 6,000 of number average molecular weight and 20 to 90% by weight of ethylene oxide contents. The isocyanate may be one or more selected from the group consisting of methylene diphenyl diisocyanate and toluene diisocyanate. And also, the polyurethane prepolymer may further comprise one or more cross-linking agents selected from the group consisting of ethylene glycol, propylene glycol, 1,3-butanediol, 1,4-butanediol, 1,5-pentanediol, 1,6-hexanediol, triethylene glycol, diethylene glycol, tetraethylene glycol, dipropylene glycol, dibutylene glycol, neopentyl glycol, 1,4-cyclohexanedimethanol, and 2-methyl-1,3-pentanediol.

The polymerization initiator may be one or more selected from the group consisting of ethylene glycol, propylene glycol, glycerin, pentaerythritol, and glycine, preferably glycine. The blowing solution may further comprise one or more surfactants selected from the group consisting of a block copolymer of ethylene oxide and propylene oxide and a silicone surfactant. And also, the drying may be performed by casting the polyurethane foam into a mold having a release liner on the bottom; and then drying at 65° C. to 75° C. for 20 minutes to 1 hour.

### Advantageous Effects

The process for preparing a polyurethane foam dressing according to the present invention includes mixing a solution obtained by dissolving an anti-inflammatory agent in the certain dispersing agent such as ethanol with a polyurethane prepolymer to obtain a drug-containing polyurethane prepolymer. The process of the present invention including said step can provide a polyurethane foam dressing in which an anti-inflammatory agent is homogeneously dispersed. Especially, in the process of the present invention, the dispersing agent can be easily removed through conventional drying methods, while maintaining not only the blowability in the foam-forming step but also the exudate-absorbing capacity of the resulting polyurethane foam dressing. Therefore, the process of the present invention makes it possible to prepare a polyurethane foam dressing in which an anti-inflammatory agent is homogeneously dispersed, having excellent blowability and exudate-absorbing capacity.

### DESCRIPTION OF DRAWINGS

FIG. 1a shows the results obtained by observing the dexibuprofen-containing polyurethane foam dressings prepared according to the present invention through a scanning electron microscope.

FIG. 1b shows the results obtained by observing the dexibuprofen-containing polyurethane foam dressings prepared by using surfactants or polyalcohols through a scanning electron microscope. A: the dexibuprofen solution in 10% PEG solution was used, B: the dexibuprofen solution in 30% Tween 80 solution was used, C: the dexibuprofen solution in 1% Poloxamer 407 solution was used.

FIG. 2 shows the appearances and sizes of the non-drug containing polyurethane foam (A) and the dexibuprofen-containing polyurethane foam (B).

FIG. 3 shows the results obtained by measuring the skin penetration rate of dexibuprofen, after applying the polyurethane foam dressing prepared according to the present invention to the skin of hairless mice.

### BEST MODE

As used herein, the expression “polyurethane foam dressing in which an anti-inflammatory agent is homogeneously dispersed” refers to a polyurethane foam dressing where a drug (i.e., an anti-inflammatory agent) is dispersed or distributed uniformly without aggregation.

The term “dexibuprofen” refers to the dextrorotatory enantiomer of ibuprofen and the chemical name thereof is (2S)-2-(4-isobutylphenyl)propionic acid.

The present invention provides a process for preparing a polyurethane foam dressing in which an anti-inflammatory agent is homogeneously dispersed, the process comprising: (a) mixing an alkaline anti-inflammatory agent having a pKa value more than pKa 7; one or more dispersing agents selected from the group consisting of methanol, ethanol, n-propanol, isopropanol, ethyl acetate and n-hexane; and a polyurethane prepolymer comprising a polyol and an isocyanate to obtain a drug-containing polyurethane prepolymer, and (b) reacting the drug-containing polyurethane prepolymer obtained in the step (a) with a blowing solution comprising water and a polymerization initiator to form a polyurethane foam and then drying the polyurethane foam.

In the process of the present invention, the step (a) may be performed by mixing a drug solution obtained by dissolving the anti-inflammatory agent in the dispersing agent with the polyurethane prepolymer. The anti-inflammatory agent may be acetaminophen, ibuprofen, or dexibuprofen, preferably dexibuprofen. The anti-inflammatory agent may be used in the respective therapeutically effective amounts.

It is preferable that the dispersing agent is used in an amount which allows to dissolve the therapeutically effective amount of the anti-inflammatory agent and not to reduce and/or lose blowability of the resulting foam dressing. For example, the dispersing agent may be used in an amount ranging from 0.2 to 20% by weight, preferably from 0.2 to 10% by weight, based on the total weight of the polyurethane prepolymer. The dispersing agent may be methanol, ethanol, n-propanol, or isopropanol, preferably ethanol.

It has been found by the present invention that the use of dexibuprofen as an anti-inflammatory agent can accomplish desired pharmacological effects with a low amount thereof (e.g., a half amount of ibuprofen) and minimize the amount of the dispersing agent such as ethanol. Especially, it has been found by the present invention that the use of dexibuprofen in combination with ethanol in the polyurethane foam dressing can maintain the equivalent blowability to a non-drug containing polyurethane foam dressing. Therefore, in an embodiment, the step (a) may be performed by mixing a drug solution obtained by dissolving dexibuprofen in ethanol with the polyurethane prepolymer. The drug solution may be obtained by dissolving dexibuprofen in ethanol in a concentration ranging from 0.005 to 50 g/ml, preferably from 0.05 to 5 g/ml, more preferably about 0.5 g/ml.

Polyols and isocyanates conventionally used in the field of the polyurethane foam dressing may be used for the polyurethane prepolymer used in the step (a). For example, the polyurethane prepolymer may be prepared with a mixture obtained by using various polyols and isocyanates disclosed in the art (e.g., Korean Patent Publication No. 2002-0046619). For example, a copolymer of ethylene oxide and propylene oxide having 500 to 6,000 of number average molecular weight and 20 to 90% by weight of ethylene oxide contents may be used as the polyol. And also, the isocyanate may be one or more selected from the group consisting of methylene diphenyl diisocyanate and toluene diisocyanate. The polyol and the isocyanate may be used in a weight ratio ranging from 3:1 to 5:1, but not limited thereto. And also, the polyurethane prepolymer may further comprise one or more cross-linking agents selected from the group consisting of ethylene glycol, propylene glycol, 1,3-butanediol, 1,4-butanediol, 1,5-pentanediol, 1,6-hexanediol, triethylene glycol, diethylene glycol, tetraethylene glycol, dipropylene glycol, dibutylene glycol, neopentyl glycol, 1,4-cyclohexanedimethanol, and 2-methyl-1,3-pentanediol. The amount of the cross-linking agent is not particularly limited, as long as desired cross-linking effects can be obtained.

The blowing solution used in the step (b) includes water (e.g., deionized water, etc.) which functions as a blowing agent; and a polymerization initiator. The addition of the blowing solution gives a polyurethane foam through the pores formed by gas generation. As described in the above, the blowability in the foam-forming step can be effectively maintained, although the dispersing agent is added. The water may be used in an amount for accomplishing desired blow, e.g., in an amount ranging from 0.1 to 1 part by weight, based on 1 part by weight of the drug-containing polyurethane prepolymer obtained in the step (a). The polymerization initiator may be one or more selected from the group consisting of ethylene glycol, propylene glycol, glycerin, pentaerythritol, and glycine. It has been also found by the present invention that the use of glycine as the polymerization initiator can accomplish a moisturizing effect, in addition to the polymerization initiating effect. Therefore, glycine may be preferably used as the polymerization initiator. If necessary, the blowing solution may further comprise one or more surfactants selected from the group consisting of a block copolymer of ethylene oxide and propylene oxide (e.g., Poloxamer™) and a silicone surfactant (e.g., L-688, L-626, etc.). The amount thereof may be determined appropriately by a skilled person in the art.

The process of the present invention includes drying the polyurethane foam obtained by said polymerization. The drying may be performed by casting the polyurethane foam into a mold having a release liner on the bottom; and then drying at 65° C. to 75° C. for 20 minutes to 1 hour, preferably at about 70° C. for about 30 minutes. As described in the above, the dispersing agent can be easily removed through conventional drying methods; and allows to effectively maintain the exudate-absorbing capacity of the resulting polyurethane foam dressing, even after performing the drying step. The release liner, a backing membrane which does not contact with skin, may be formed by using non-drug-absorbable and flexible materials. For the release liner, conventional films, such as polyolefin, polyether, polyester, or polyurethane, etc., coated with silicon. The polyurethane foam dressing obtained according to the process of the present invention may be formulated to contain e.g., dexibuprofen in the concentration ranging from 0.01 to 2.5 mg/cm² per unit area. The concentration thereof may be controlled by appropriately selecting the mold size, the thickness of the polyurethane foam dressing (i.e., the thickness of the formulation except for the release liner), etc.

The present invention will be described in further detail with reference to the following examples and experimental examples. These examples and experimental examples are for illustrative purposes only and are not intended to limit the scope of the present invention.

### Example 1: Preparation and Evaluation of Polyurethane Foam Dressings

(1) Preparation of the Polyurethane Prepolymer

Polyether polyol (Molecular weight: 4,800, VORANOL™, The Dow Chemical Company) (39.81 g) was heated to 70° C., under stirring at 150 rpm. 2,4-Toluene diisocyanate (9.6 g), ethylene glycol (0.24 g), and 1,4-butanediol (0.35 g) were added thereto. The reaction mixture was stirred for 4 hours to prepare the polyurethane prepolymer. (Amount of NCO in the polyurethane prepolymer (%)=14%)

(2) Preparation and Evaluation of Dexibuprofen-Containing Polyurethane Foam Dressings

Dexibuprofen was dissolved in ethanol in the concentration of 0.5 g/ml. 0 ml, 0.02 ml, 0.1 ml, or 0.2 ml of the resulting solution was respectively mixed with the polyurethane prepolymer prepared in the above. The blowing solution (containing 33.4 g of deionized water, 15 g glycine, and 3.34 g of Poloxamer 188) was added to the respective mixtures. The mixtures were stirred at 150 rpm for 15 seconds to form polyurethane foams. The resulting polyurethane foams were casted into a mold having the 10 cm×10 cm size on the silicon-coated release liner and then maturated for 10 minutes. The obtained polyurethane foams were dried at 70° C. for 30 minutes to prepare the polyurethane foam dressings, each containing 0 mg/cm², 0.1 mg/cm², 0.5 mg/cm², and 1.0 mg/cm² (corresponding to 0, 10, 50, and 100 mg per unit foam dressing formulation, respectively) of dexibuprofen.

For comparison, the polyurethane foam dressing containing 0.5 mg/cm² of dexibuprofen were prepared in accordance with the same procedures as in the above, except for mixing the solutions (each being 0.1 ml) (which were obtained by dissolving dexibuprofen in a 10% PEG solution, a 30% Tween 80 solution, or a 1% Poloxamer 407 solution in the concentration of 0.5 g/ml) with the polyurethane prepolymer prepared in the above.

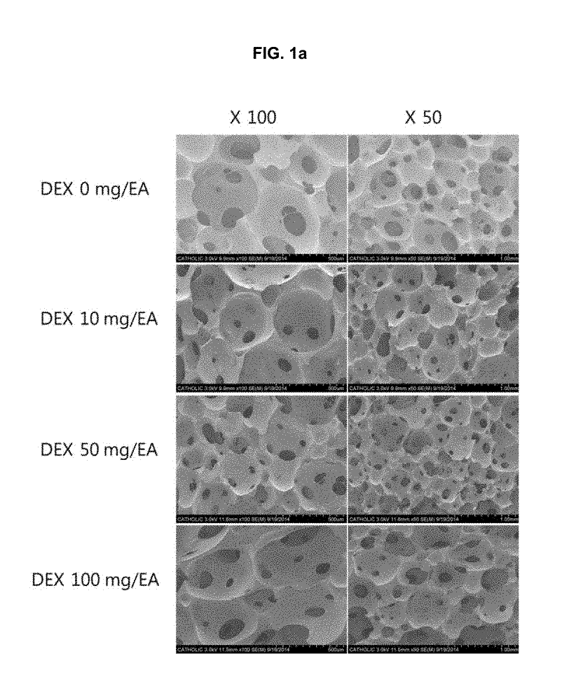

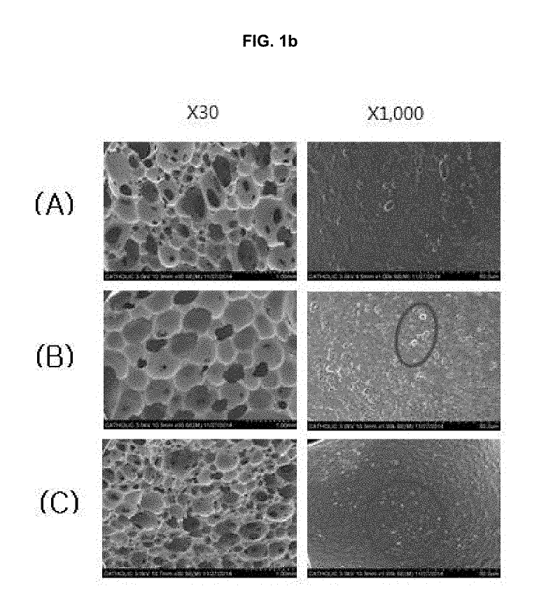

FIGS. 1a and 1b show the results obtained by observing the obtained polyurethane foam dressings through a scanning electron microscope. The polyurethane foam dressings prepared according to the present invention have similar appearances in the pore shapes and the sizes thereof, to the non-drug containing polyurethane foam dressing, without showing any drug aggregation (FIG. 1a). However, in the polyurethane foam dressings prepared by using surfactants or polyalcohols, the pores were not formed uniformly; and drug aggregations were observed (FIG. 1b).

And also, the polyurethane foam dressings prepared by using the solution of dexibuprofen in ethanol were cut into the pieces having 1 cm² size, which were then added to phosphate buffered saline. The exudate-absorbing capacities thereof were evaluated through measuring the weights of the foam dressings before and after the absorptions of phosphate buffered saline. The results are shown in the following table 1.

TABLE 1

| | 0 mg/EA | 10 mg/EA | 50 mg/EA | 100 mg/EA |
|---|---|---|---|---|
| Before the | 0.42 ± 0.03 | 0.43 ± 0.04 | 0.38 ± 0.02 | 0.42 ± 0.02 |
| absorption (g) | | | | |
| After the | 0.76 ± 0.03 | 0.77 ± 0.02 | 0.75 ± 0.03 | 0.70 ± 0.01 |
| absorption (g) | | | | |

As shown in Table 1, the polyurethane foam dressings prepared according to the present invention showed equivalent exudate-absorbing capacities to the non-drug containing polyurethane foam dressing.

(3) Preparation of Polyurethane Foam Dressings and Evaluation of Blowabilities

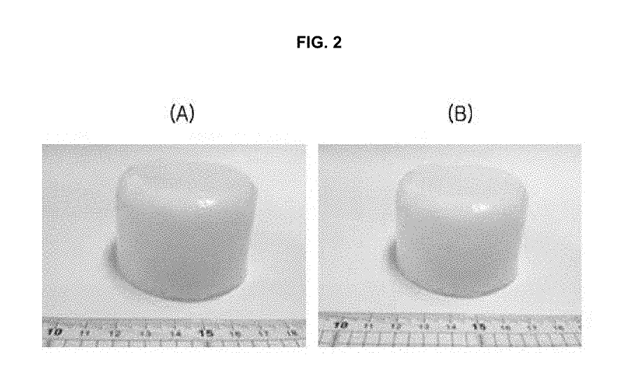

Dexibuprofen was dissolved in ethanol in the concentration of 0.5 g/ml. 0 ml or 0.5 ml of the resulting solution was respectively mixed with the polyurethane prepolymer prepared in the above. The blowing solution (containing 33.4 g of deionized water, 15 g glycine, and 3.34 g of Poloxamer 188) was added to the respective mixtures. The mixtures were stirred at 150 rpm for 15 seconds to form polyurethane foams. The resulting polyurethane foams were casted into a mold having the 10 cm×10 cm size on the silicon-coated release liner and then maturated for 10 minutes. The obtained polyurethane foams were dried for 30 minutes at a 70° C. incubator to remove ethanol, so as to obtain the solidified polyurethane foam. FIG. 2 shows the appearances and sizes of the respective polyurethane foams. As shown in FIG. 2, the polyurethane foams prepared by using the solution of dexibuprofen in ethanol showed equivalent blowability to the non-drug containing polyurethane foam.

### Example 2: Preparation of Polyurethane Foam Dressing

Ibuprofen was dissolved in ethanol in the concentration of 0.5 g/ml. 1 ml of the resulting solution was mixed with the polyurethane prepolymer prepared in accordance with the same procedures as in Example 1 (1). The blowing solution (containing 33.4 g of deionized water, 15 g glycine, and 3.34 g of Poloxamer 188) was added to the mixture, which was then stirred at 150 rpm for 15 seconds to form a polyurethane foam. The resulting polyurethane foam was casted into a mold having the 10 cm×10 cm size on the silicon-coated release liner and then maturated for 10 minutes. The obtained polyurethane foam was dried at 70° C. for 30 minutes to prepare the polyurethane foam dressing containing 5 mg/cm² of ibuprofen.

Experimental Example 1: Measurement of Skin Penetration of the Polyurethane Foam Dressing

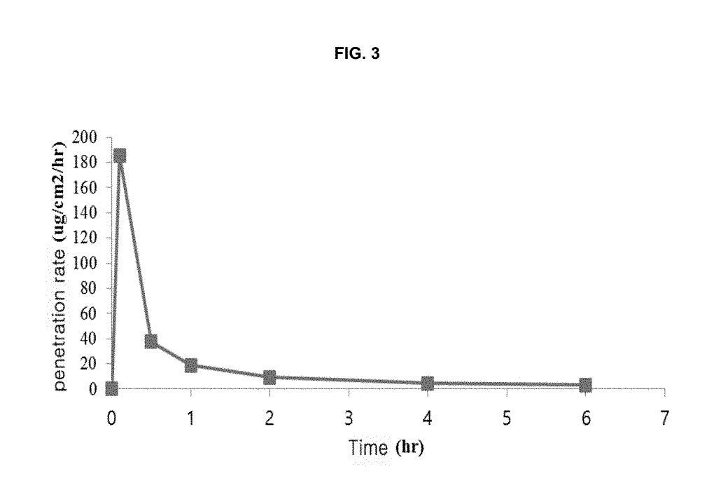

The polyurethane foam dressing containing 0.1 mg/cm² of dexibuprofen, which was prepared in the above, was cut into the pieces having 2 cm² size, which were then applied onto the skin of about eight-week old hairless mice (Orient Bio Inc., Korea). The skin penetration rate was measured at 37° C., using a pH 6.8 phosphate buffer as a receiver medium. The aliquots were taken from the receiver medium at 0 minute, 10 minutes, 30 minutes, 1 hour, 2 hours, 4 hours, and 6 hours; and the concentrations of dexibuprofen therein were measured with high performance liquid chromatography (HPLC). The results are shown in FIG. 3.

As shown in FIG. 3, the dexibuprofen-containing polyurethane foam dressing prepared according to the present invention exhibited rapid initial drug penetration, showing that the drug more than 40% is penetrated within 30 minutes. Considering that dexibuprofen, one of the non-steroidal anti-inflammatory agents, should exhibit high initial release rate so as to be rapidly absorbed in the wound site for pain relief, it is expected that said polyurethane foam dressing can be very usefully applied.

1. A process for preparing a polyurethane foam dressing in which an anti-inflammatory agent is homogeneously dispersed, the process comprising:
(a) mixing an alkaline anti-inflammatory agent having a pKa value more than pKa 7; one or more dispersing agents selected from the group consisting of methanol, ethanol, n-propanol, isopropanol, ethyl acetate and n-hexane; and a polyurethane prepolymer comprising a polyol and an isocyanate to obtain a drug-containing polyurethane prepolymer, and
(b) reacting the drug-containing polyurethane prepolymer obtained in the step (a) with a blowing solution comprising water and a polymerization initiator to form a polyurethane foam and then drying the polyurethane foam.

2. The process according to claim 1, wherein the step (a) is performed by mixing a drug solution obtained by dissolving the anti-inflammatory agent in the dispersing agent with the polyurethane prepolymer.

3. The process according to claim 1, wherein the anti-inflammatory agent is acetaminophen, ibuprofen, or dexibuprofen.

4. The process according to claim 3, wherein the anti-inflammatory agent is dexibuprofen.

5. The process according to claim 1, wherein the dispersing agent is used in an amount ranging from 0.2 to 20% by weight, based on the total weight of the polyurethane prepolymer.

6. The process according to claim 1, wherein the dispersing agent is ethanol.

7. The process according to claim 1, wherein the step (a) is performed by mixing a drug solution obtained by dissolving dexibuprofen in ethanol with the polyurethane prepolymer.

8. The process according to claim 7, wherein the drug solution is obtained by dissolving dexibuprofen in ethanol in a concentration ranging from 0.005 to 50 g/ml.

9. The process according to claim 1, wherein the polyol is a copolymer of ethylene oxide and propylene oxide having 500 to 6,000 of number average molecular weight and 20 to 90% by weight of ethylene oxide contents.

10. The process according to claim 1, wherein the isocyanate is one or more selected from the group consisting of methylene diphenyl diisocyanate and toluene diisocyanate.

11. The process according claim 1, wherein the polyurethane prepolymer further comprises one or more cross-linking agents selected from the group consisting of ethylene glycol, propylene glycol, 1,3-butanediol, 1,4-butanediol, 1,5-pentanediol, 1,6-hexanediol, triethylene glycol, diethylene glycol, tetraethylene glycol, dipropylene glycol, dibutylene glycol, neopentyl glycol, 1,4-cyclohexanedimethanol, and 2-methyl-1,3-pentanediol.

12. The process according to claim 1, wherein the polymerization initiator is one or more selected from the group consisting of ethylene glycol, propylene glycol, glycerin, pentaerythritol, and glycine.

13. The process according to claim 12, wherein the polymerization initiator is glycine.

14. The process according to claim 1, wherein the blowing solution further comprises one or more surfactants selected from the group consisting of a block copolymer of ethylene oxide and propylene oxide and a silicone surfactant.

15. The process according to claim 1, wherein the drying is performed by casting the polyurethane foam on a mold having a release liner on the bottom; and then drying at 65° C. to 75° C. for 20 minutes to 1 hour.
